(19) United States
(12) Patent Application Publication
(10) Pub. No.: US 2007/0224662 A1
(43) Pub. Date: Sep. 27, 2007

(54) Post-translational modification of proteins in cell-free expression systems

(76) Inventors: Jun Luo, Somerville, MA (US); George Martin, Berkley, CA (US)

Correspondence Address: ROCHE DIAGNOSTICS OPERATIONS INC., 9115 Hague Road, Indianapolis, IN, 46250-0457, US

(21) Appl. No.: 11/303,901

(22) Filed: Dec. 16, 2005

Related U.S. Application Data
(60) Provisional application No. 60/637,404, filed on Dec. 17, 2004.

(51) Int. Cl. C12P 21/06; C12N 9/12

(52) U.S. Cl. 435/69.1; 435/194

(57) ABSTRACT

Disclosed is a method for post-translational modification of a recombinant protein in a cell-free expression system. The method comprises co-expressing the enzyme responsible for the post-translational modification along with the target protein in a prokaryotic based in vitro expression system. In one embodiment the expression system further comprises a eukaryotic cell extract, and in an alternative embodiment the target protein is co-expressed with a modified kinase that is constitutively active. In particular, a method for post-translational modification of a highly active MAPK 14 is described.

### FIELD OF THE INVENTION

The present invention relates to the field of cell-free protein expression, and more particularly, to a method for post-translational modification of recombinant proteins.

### BACKGROUND OF THE INVENTION

Typically, proteins that require post-translational modifications are produced solely in eukaryotic expression systems. There are examples whereby the protein is expressed and purified followed by modification in vitro. This is typically how active protein kinases are commercially prepared. An example of in vitro glycosylation of purified proteins is Neose.

Post-translational modification of recombinant proteins is an inefficient process that normally does not occur in in vitro translation systems. In vitro translation systems, particularly bacterial-based in vitro translation systems, lack many of the enzymes that are required for these post-translational modifications. Examples of post-translational modifications that commonly occur in cells but not in in vitro expression systems include, but are not limited to, phosphorylation, glycosylation, proteolysis, and palmitoylation.

Post-translational modification is required for the function of many biologically important proteins. Modification may result in the activation, localization, and solubilization of proteins. The present invention provides a means for these post-translational modifications to occur in vitro with very high efficiency and permits preparative post-translational modifications to occur as the protein is being synthesized.

Signal transduction via mitogen activated protein (MAP) kinases plays a key role in a variety of cellular responses, including proliferation, differentiation, and cell death. MAP kinases (MAPK) mediate signal transduction from the cell surface to the nucleus via phosphorylation cascades. The three major MAP kinase pathways include the extracellular-signal regulated kinase (ERK, also known as MAP kinase), c-jun N-terminal kinase (JNK, also known as stress activated protein kinase-1 (SAPK1)) and p38 MAPK (also known as SAPK2/R). In general, ERK1 and ERK2 are key transducers of proliferation signals and are often activated by mitogens. In contrast, Each MAPK cascade consists of a core MAPK module, which has no less than three enzymes activated in series: 1) a MAPK, 2) an immediate upstream kinase (known as Mitogen Activated Protein Kinase Kinase or MAPKK), and 3) an additional kinase upstream of the MAPKK (known as Mitogen Activated Protein Kinase Kinase Kinase or MAPKKK). These regulatory cascades not only convey information to the target effectors, but also coordinate incoming information from parallel signaling pathways. These mechanisms allow for signal amplification and generate a threshold subject to multiple activation cascades.

The interactions between MAP kinase and its immediate upstream kinase (MAPKK) are highly specific: for instance, MAP kinases are phosphorylated solely by MEK 1 and 2; p38 MAP kinase is selectively activated by MKK 3, 4 and 6, while JNK is activated by MKK 4, 6 and 7 in most conditions. The specificity is less clearly defined for elements upstream of the MAPKK modular level. For instance MAPKKK are highly promiscuous and can interact with and activate a number of down stream components. Similarly, signaling cross talk in the transmission levels between the mitogen/stress activator and the core MAPK module understandably adds more complexity to subtle differences in response despite equivalent activation. The specificity upstream of the core module may be regulated by additional components like scaffold proteins that help bring the specific components of the MAPK machinery together or keep various components from interacting with each other. A simplistic view of the MAP kinase signal transduction is presented in FIG. 1.

### SUMMARY OF THE INVENTION

It is against the above background that the present invention provides certain unobvious advantages and advancements over the prior art. In particular, the inventor has recognized a need for improvements in preparative post-translational modifications of proteins in cell-free expression systems.

Although the present invention is not limited to specific advantages or functionality, it is noted that the present invention provides a method whereby preparative amounts of protein can be produced with correct and homogeneous post-translational modification. In particular, the present invention provides a method for the in vitro production of a properly phosphorylated protein kinase.

In accordance with one embodiment of the present invention, co-expression of the target protein and the enzyme responsible for the post-translational modification of the target protein in a prokaryotic based in vitro expression system that includes a eukaryotic cell extract fraction, provides high quality and specific modification of the target protein. In accordance with one embodiment the target protein is a kinase that is activated by posttranslational modification, and in one embodiment the target protein is MAPK 14.

In an alternative embodiment, co-expression of the target protein and the enzyme responsible for the post-translational modification of the target protein in a prokaryotic based in vitro expression system is conducted in the absence of a eukaryotic cell extract fraction and also provides high quality and specific modification of the target protein. In this embodiment the target protein is co-expressed with the enzyme responsible for the post-translational modification of the target protein, wherein the enzyme has been modified relative to its wild type counterpart to be contitutively active. In accordance with this invention “a constitutively active enzyme” is an enzyme that exhibits the enzymatic characteristics of the native activated enzyme without requiring post-transitional modification of the expressed protein.

In accordance with one embodiment the expression system described herein is used to produce a highly active MAPK 14 (mitogen-activated protein kinase) preparation. By use of the novel kinase activation of the present invention, it is possible to produce large amounts of active kinase in a highly purified state, and as a result, it provides reagents that can potentially be used to screen for novel substances useful for treating or preventing disease.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 is a schematic drawing showing the MAP kinase activation cascade.

FIG. 2 is a Western blot of 3 sets of RTS 500 in vitro transcription/translation reactions using an anti-phospho-p38 antibody. Lane 1 represents a reaction run using only MAPK14 DNA, lane 2 represents a reaction run using MAPK14 DNA plus HEK 293 extract and lane 3 represents a reaction run with MAPK14 DNA plus HEK 293 extract and MKK6 DNA.

FIGS. 3A-3C represent Western blots of a series of in vitro transcription/translation reactions conducted in the presence of an HEK 293 extract, MAPK14 DNA and decreasing amounts of MKK6 DNA. FIG. 3A is a Western blot demonstrating the decreased amount of MKK6 protein produced as the plasmid levels were decreased in the reactions (see lanes 3-7). FIG. 3B is a Western blot demonstrating the decreased phosphorylated form of MAPK14 (i.e. the activated form) as the level of MKK6 protein decreases. MAPK14 was expressed with a histidine tag and antibodies to that tag were used to measure total expression of the MAPK14 protein in the RTS 500 reactions. FIG. 3C is a Western blot demonstrating that total levels of MAPK14 did not vary substantially between individual reactions (see lanes 2-7 of FIG. 3C; lane 1 representing his-tagged molecular markers).

FIG. 4 represents a mass spectrometry analysis of the tryptic fragment 174-HTDDEMTGYVATR-186 (SEQ ID NO: 20) produced from inactive and active MAPK14. Active MAPK14 produced in accordance with the present invention is phosphorylated on the expected tryptic fragment.

FIG. 5 represents a mass spectrometry analysis of a kinase activity reaction conducted with active and inactive MAPK14 protein. Only activated MAPK14 successfully phosphorylated the MAPK14 substrate KRELVEPLTPSGEAPNQALLR (SEQ ID NO: 21), shifting the molecular weight of the peptide from 2313 Da to 2392.6 Da.

### DETAILED DESCRIPTION OF THE INVENTION

Definitions

In describing and claiming the invention, the following terminology will be used in accordance with the definitions set forth below.

The term “activated” as used in the context of a protein enzyme refers to a purified recombinant protein that exhibits substantial enzymatic activity. For example, an activated kinase is a kinase that has been phosphorylated resulting in the kinase being capable of phosphorylating its target in an amount statistically significant versus the corresponding non-phosphorylated kinase.

The term “mitogen activated protein kinase” refers to extracellular signal-regulated protein kinases that effect processes in the cytoplasm, the nucleus and the cytoskeleton and can induce proliferation or enhance differentiation. They have been referenced in the literature as MAPKs or ERKs. For this application the abbreviation MAPK will be used, with the individual family members identified by a numerical designation following the MAPK abbreviation (e.g. MAPK14). A generic designation of one MAPK family (e.g. MAPK14) is intended to encompass all know members of that kinase family (e.g. all known variants of the MAPK14 amino acid sequence of SEQ ID NO: 12).

The term “mitogen activated protein kinase kinase” refers to extracellular signal-regulated protein kinases that are the upstream activators of the MAP kinases. They have been referenced in the literature as MEKs, MAPKKs and MKKs. For this application the abbreviation MKK will be used, with the individual family members identified by a numerical designation following the MKK abbreviation (e.g. MKK2). A generic designation of one MKK family (e.g. MKK6) is intended to encompass all know members of that kinase family (e.g. all known variants of the MKK 6 amino acid sequence of SEQ ID NO: 18).

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

A “linker” is a molecule or group of molecules that are bound at one end to a first component and bound at a second end to a second component, thus attaching the first and second components to one another by a space determined by the length of the linker. Linkers may further supply a labile linkage that allows for subsequent separation of the first and second components. Labile linkages include photocleavable groups, acid-labile moieties, base-labile moieties and enzyme-cleavable groups.

In order that the invention may be more readily understood, reference is made to the following embodiments and examples, which are intended to illustrate the invention, but not limit the scope thereof.

### Embodiments

In accordance with one embodiment of the invention a method for expressing an activated recombinant target kinase through the use of a cell free expression system is provided. Advantageously, the method allows for the production of an active kinase without requiring a purification step. Once the activated kinase is synthesized, the activated kinase can be subsequently purified using standard techniques. In one embodiment the recombinantly produced activated kinases are synthesized having a peptide tag (such as a six amino acid histidine terminal extension) that assists in the purification of the recombinantly produced activated kinase. In one embodiment the peptide tag can be bound the terminus of the kinase via a linker, and in one embodiment the linker is a labile linker.

In accordance with one embodiment of the present invention, both the target kinase to be activated and the enzyme responsible for the post-translational modification that activates the target kinase are co-expression in an in vitro expression system to provide a high quality and specific modification of the target protein resulting in activation of the expressed target protein kinase. More particularly, the posttranslational modification comprises phosphorylation of the target kinase. In accordance with one embodiment the target kinase is a MAP kinase and the enzyme responsible for the phosphorylation of the MAP kinase is a MAP kinase kinase (MKK), wherein activation of the MAP kinase results in phosphorylation of MAPK at two sites. The two activating phosphorylation sites are a tyrosine and a threonine (e.g. Thr-180 and Tyr-182 of MAPK14; SEQ ID NO: 12).

In accordance with one embodiment the MAP kinase comprises an amino acid sequence selected from the group consisting of MAPK1/ERK2 (SEQ ID NO: 1), MAPK3/ERK1 (SEQ ID NO: 2), MAPK4/ERK4 (SEQ ID NO: 3), MAPK6/ERK3 (SEQ ID NO: 4), MAPK7/ERK5 (SEQ ID NO: 5), MAPK8/JNK1 (SEQ ID NO: 6), (MAPK9/JNK2 (SEQ ID NO: 7), MAPK10/JNK3 (SEQ ID NO: 8), MAPK11/p38 beta (SEQ ID NO: 9), MAPK12/p38 gamma (SEQ ID NO: 10), MAPK13/p38 delta (SEQ ID NO: 11) and MAPK14 (SEQ ID NO: 12). In one embodiment the enzyme responsible for the post-translational modification of the target kinase comprises an amino acid sequence selected from the group consisting of MKK1 (SEQ ID NO: 13), MKK2 (SEQ ID NO: 14), MKK3 (SEQ ID NO; 15), MKK4 (SEQ ID NO: 16), MKK5 (SEQ ID NO: 17), MKK6 (SEQ ID NO:18), modified MKK6 (SEQ ID NO: 22) and MKK7 (SEQ ID NO:19).

In one embodiment a MAP kinase, comprising an amino acid sequence selected from the group consisting of MAPK1/ERK2 (SEQ ID NO: 1), MAPK3/ERK1 (SEQ ID NO: 2), MAPK4/ERK4 (SEQ ID NO: 3), MAPK6/ERK3 (SEQ ID NO: 4), MAPK7/ERK5 (SEQ ID NO: 5), MAPK11/p38 beta (SEQ ID NO: 9), MAPK12/p38 gamma (SEQ ID NO: 10), MAPK13/p38 delta (SEQ ID NO: 11) and MAPK14 (SEQ ID NO: 12) is co-expressed with a MKK comprising a sequence selected from the group consisting of MKK3 (SEQ ID NO; 15), MKK4 (SEQ ID NO: 16) and MKK6 (SEQ ID NO:18 or SEQ ID NO: 22). In another embodiment a MAP kinase, comprising an amino acid sequence selected from the group consisting of MAPK8/JNK1 (SEQ ID NO: 6), (MAPK9/JNK2 (SEQ ID NO: 7), MAPK10/JNK3 (SEQ ID NO: 8), is co-expressed with an MKK comprising a sequence selected from the group consisting of MKK4 (SEQ ID NO: 16) and MKK7 (SEQ ID NO: 19). In another embodiment a MAP kinase, comprising an amino acid sequence selected from the group consisting of MAPK1/ERK2 (SEQ ID NO: 1), MAPK3/ERK1 (SEQ ID NO: 2), MAPK4/ERK4 (SEQ ID NO: 3), MAPK6/ERK3 (SEQ ID NO: 4) and MAPK14 (SEQ ID NO: 12) is co-expressed with a MKK comprising a sequence selected from the group consisting of MKK1 (SEQ ID NO; 13) and MKK2 (SEQ ID NO: 14). In another embodiment a MAP kinase, comprising an amino acid sequence selected from the group consisting of MAPK7/ERK5 (SEQ ID NO: 5) is co-expressed with a MKK comprising a sequence selected from the group consisting of MKK5 (SEQ ID NO: 17). In another embodiment a MAP kinase, comprising an amino acid sequence selected from the group consisting of MAPK8/JNK1 (SEQ ID NO: 6), (MAPK9/JNK2 (SEQ ID NO: 7), MAPK10/JNK3 (SEQ ID NO: 8), MAPK11/p38 beta (SEQ ID NO: 9), MAPK12/p38 gamma (SEQ ID NO: 10), MAPK13/p38 delta (SEQ ID NO: 11) and MAPK14 (SEQ ID NO: 12) is co-expressed with a MKK6 (SEQ ID NO:18). In another embodiment MAPK14 (SEQ ID NO: 12) is co-expressed with a MKK6 (SEQ ID NO: 18 or SEQ ID NO: 22). Nucleic acid sequences encoding the various native MAPKs and MKKs have been disclosed in the literature are known to those skilled in the art.

In one embodiment the expression system used to co-express the target and activating kinases comprises a prokaryotic based in vitro expression system that has been modified by the inclusion of a eukaryotic cell extract. In accordance with one embodiment the components comprise an E. coli cell extract and a eukaryotic cell extract. In accordance with one embodiment an expression reagent is provided comprising a prokaryotic extract and a eukaryotic cell extract in a ratio of about 20:1 to about 3:1 and in one embodiment the ratio of prokaryotic extract to eukaryotic extract is about 10:1 to about 4:1 and in a further embodiment the ratio of prokaryotic extract to eukaryotic extract is about 6:1 to about 5:1. In one embodiment the prokaryotic expression system comprises the Rapid Translation System (RTS), commercially available from Roche Diagnostics Corporation, Indianapolis, Ind., USA, that has been modified to include a eukaryotic cell extract. In one embodiment the eukaryotic cell extract is prepared from mammalian cells, and more particularly human cells, and in one embodiment the extract is derived from HEK 293 cells.

The method of producing an activated target kinase comprises the steps of co-expressing a gene encoding the target kinase with a gene that encodes an enzyme (e.g. an “activating kinase”) for activation of the target kinase. The activating kinase can be co-expressed in a prokaryotic based expression system that has been modified to include eukaryotic cell extract components. Alternatively, the co-expression of the target and activating kinases can take place using only a prokaryotic based expression system (devoid of eukaryotic cell components), if a modified activating kinase is used, wherein the activating kinase is constitutively active (i.e. does not need to be post-translationally modified to have kinase activity). In this latter embodiment the modified activating kinase has been modified by one or more amino acid substitutions, wherein the serine and threonine residues that are normally phosphorylated in the active form of the kinase have been replaced with glutamic acid residues.

In one embodiment the activating kinase used to activate the target kinase is initially expressed in an inactive state, and the expressed non-phosphorylated activating kinase is phosphorylated, and thus activated, within the expression system reaction mixture, without requiring the purification of the expressed activating kinase. In this embodiment, the expression system comprises components from a eukaryotic cell extract. In accordance with one embodiment the method of producing an active recombinant target kinase comprises the steps of providing a reaction mixture comprising a prokaryotic cell extract, a eukaryotic cell extract, a gene encoding the target kinase, and a gene encoding an activating kinase and co-expressing the target kinase and activating kinase. Co-expression of the target and activating kinases, using the modified prokaryotic expression system of the present invention, results in the production of the target kinase in an active form. The activated target kinase can then be subsequently purified from the expression system reagents.

In accordance with one embodiment the target kinase is a MAP kinase, and in one embodiment the target kinase is selected from the group consisting of MAPK1/ERK2, MAPK3/ERK1, MAPK4/ERK4, MAPK6/ERK3, MAPK7/ERK5, MAPK8/JNK1, MAPK9/JNK2, MAPK10/JNK3, MAPK11/p38 beta, MAPK12/p38 gamma, MAPK13/p38 delta and MAPK14. In one embodiment the activating kinase is an MKK, and in on embodiment the MKK is selected from the group consisting of MKK1, MKK2, MKK3, MKK4, MKK5, MKK6 and MKK7. In accordance with one embodiment the target kinase is selected from the group consisting of MAPK8/JNK1, MAPK9/JNK2, MAPK10/JNK3 and MAPK14 and the activating kinase is selected from the group consisting of MKK3, MKK4 and MKK6. In accordance with one embodiment the target kinase is selected from the group consisting of MAPK8, MAPK9, MAPK10, MAPK11, MAPK12, MAPK13 and MAPK14 and the activating kinase is MKK6.

In accordance with one embodiment the gene encoding the target kinase comprises a nucleic acid sequence selected from the group consisting of SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 40 which encode the kinases MAPK6, MAPK8, MAPK9, MAPK10, MAPK14, MAPK11, MAPK12, MAPK13, and MAPK3, respectively. In one embodiment the gene encoding the activating kinase comprises the sequence of SEQ ID NO: 23, which encodes for MKK6. In one embodiment the gene encoding the target kinase comprises a nucleic acid sequence selected from the group consisting of SEQ ID NO: 26, SEQ ID NO: 27, SEQ ID NO: 28, and SEQ ID NO: 29 and the gene encoding the activating kinase comprises the sequence of SEQ ID NO: 23. In one embodiment the gene encoding the target kinase comprises the sequence of SEQ ID NO: 29 and the gene encoding the activating kinase comprises the sequence of SEQ ID NO: 23. In one embodiment the target kinase gene comprises the sequence of SEQ ID NO: 29 and a peptide tag bound the carboxy terminus of the kinase. In a further embodiment, the sequence of SEQ ID NO: 29 further comprises a peptide tag bound the carboxy terminus of the kinase via a labile linker.

In accordance with one embodiment the activating kinase comprises a kinase that has been modified such that the recombinantly expressed activating kinase is expressed as a constitutively active form. More particularly, the activating kinase has been modified to substitute glutamic acid residues for the serine and threnine residues that are phosphorylated in the activated form of the activating kinase. In accordance with one embodiment the modified activating kinase is MKK6, wherein the modified MKK6 (mMKK6) comprises the sequence of SEQ ID NO: 22, having the serine residue at position 207 and the threonine at position 211 of the wild type sequence (SEQ ID NO: 18) replaced with glutamic acids.

In accordance with one embodiment a method for expressing an activated target kinase in a cell free expression system is provided using a constitutively active activating kinase. In this embodiment the amino acid sequence of the activating kinase has been modified relative to its wild type sequence to substitute glutamic acid residues for the serine and/or threonine residues that are phosphorylated in the activated state. In accordance with this embodiment the in vitro, cell free expression of the target kinase, in an activated form, can be conducted in the absence of eukaryotic cell components. More particularly the method comprises the steps of providing a reaction mixture comprising a prokaryotic cell extract, and co-expressing the target kinase and the constitutively active activating kinase to produce the activated form of the target kinase. Thus the activated target kinase is synthesized in vitro within the expression system reaction mixture and without requiring purification of the expressed activated target kinase. In accordance with one embodiment the target kinase is selected from the group consisting of MAPK8, MAPK9, MAPK10, MAPK11, MAPK12, MAPK13 and MAPK14 and the modified activating kinase is selected from the group consisting of mMKK3, mMKK4 and mMKK6. In this embodiment, an active MAPK is obtained when the MAPK is co-expressed with the mutated version of MKK without requiring the presence of a eukaryotic cell extract. In one embodiment the target kinase is selected from the group consisting of MAPK8, MAPK9, MAPK10, MAPK11, MAPK12, MAPK13 and MAPK14 and the modified activating kinase is mMKK6. In another embodiment the target kinase is MAPK14 and the modified activating kinase is mMKK6.

In accordance with one embodiment the gene encoding the target kinase comprises a nucleic acid sequence selected from the group consisting of SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 40. In one embodiment the gene encoding the constitutively active activating kinase comprises the sequence of SEQ ID NO: 39. In one embodiment the gene encoding the target kinase comprises a nucleic acid sequence selected from the group consisting of SEQ ID NO: 26, SEQ ID NO: 27, SEQ ID NO: 28, and SEQ ID NO: 29 and the gene encoding the constitutively active activating kinase comprises the sequence of SEQ ID NO: 39. In one embodiment the gene encoding the target kinase comprises the sequence of SEQ ID NO: 29 and the gene encoding the constitutively active activating kinase comprises the sequence of SEQ ID NO: 39. In one embodiment the target kinase gene comprises the sequence of SEQ ID NO: 29 and a peptide tag bound the carboxy terminus of the kinase. In a further embodiment, the sequence of SEQ ID NO: 29 further comprises a peptide tag bound the carboxy terminus of the kinase via a labile linker.

In accordance with one embodiment, the activated target kinase produced in accordance with the present invention has a significant level of activity relative to a negative control (e.g. recombinantly expressing the target kinase without co-expression of the activating kinase). In accordance with one embodiment the activated kinase has at lease 50% of the activity of the corresponding native activated kinase purified from a cell population. In accordance with one embodiment the activated kinase has at lease 75% of the activity of the corresponding native activated kinase purified from a cell population. In another embodiment the in vitro synthesized kinase has at least 90% of the activity of the corresponding activated kinase purified from a cell population. By use of the novel kinase activation procedure of the present invention, it is possible to produce large amounts of active kinase having a desired high level of purity, and as a result, it provides possibilities for screening novel substances for treating or preventing disease.

In accordance with one embodiment a kit is provided for expressing activated recombinant proteins. The kit comprises an expression reagent comprising an activated MKK. In accordance with one embodiment the kit comprises an expression reagent comprising a prokaryotic and eukaryotic extract. In one embodiment the prokaryotic and eukaryotic extracts are present in a ratio of about 10:1 to about 4:1. In accordance with one embodiment the expression reagent comprises an E. coli cell extract and a eukaryotic cell extract. In one embodiment the kit further comprises buffers, amino acids, enzymes and reagents for transcribing and translating gene sequences. In one embodiment the kit further comprises expression vectors for inserting a gene of interest. In one embodiment the kit further comprises a plasmid encoding an MKK protein.

In accordance with one embodiment a kit is provided comprising a prokaryotic expression system that has been modified by the inclusion of a eukaryotic cell extract. In one embodiment the prokaryotic system comprises an E. coli extract, and in on embodiment the prokaryotic expression system comprises the Rapid Translation System (RTS), commercially available from Roche Diagnostics Corporation, Indianapolis, Ind., USA, that has been modified to include a eukaryotic cell extract. A description of the RTS system is provided in U.S. Pat. No. 6,518,058, the disclosure of which is incorporated herein. In one embodiment the eukaryotic extract is derived from mammalian cells, more particularly human cells, and in one embodiment the eukaryotic extract is prepared from HEK 293 cells. In one embodiment the kit further comprising a nucleic acid sequence encoding a target kinase selected from the group consisting of SEQ ID NO: 6, SEQ ID NO: 7, SEQ ID NO: 8, SEQ ID NO: 9, SEQ ID NO: 10, SEQ ID NO: 11, and SEQ ID NO: 12, and a nucleic acid sequence encoding an activating kinase selected from the group consisting of SEQ ID NO: 16, SEQ ID NO: 18, SEQ ID NO: 19, and SEQ ID NO: 22. In one embodiment the kit comprises a nucleic acid sequence encoding an activating kinase selected from the group consisting of SEQ ID NO: 18 and SEQ ID NO: 22. In one embodiment the kit comprises a nucleic acid sequence encoding an activating kinase selected from the group consisting of SEQ ID NO: 6, SEQ ID NO: 7, SEQ ID NO: 8, SEQ ID NO: 9, SEQ ID NO: 10, SEQ ID NO: 11, and SEQ ID NO: 12, and a nucleic acid sequence encoding an activating protein comprising the sequence of SEQ ID NO: 22.

In an alternative embodiment the kit for the in vitro production of activated kinases, comprises an expression reagent comprising a prokaryotic extract, a nucleic acid sequence comprising a sequences selected from the group consisting of SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 40, and a nucleic acid sequence comprising the sequence of SEQ ID NO: 23 or SEQ ID NO: 39. In one embodiment the nucleic acid sequence encoding the target protein comprises the nucleic acid sequence of SEQ ID NO: 29 and the nucleic acid sequence encoding the activating protein comprises the sequence of SEQ ID NO: 23.

In one embodiment the kit comprises an expression system, wherein-said system consists essentially of a prokaryotic expression system, and a nucleic acid sequence encoding an mMKK that is constitutively active (i.e. does not need to be post-translationally modified to have kinase activity). In one embodiment the nucleic acid sequence encoding the consitutively active activating kinase comprises the sequence of SEQ ID NO: 39. In one embodiment the kit is further provided with nucleic acid sequences that encode for the target kinase, wherein said nucleic acid sequences comprising a sequence selected from the group consisting of SEQ ID NO: 26, SEQ ID NO: 27, SEQ ID NO: 28, and SEQ ID NO: 29. In one embodiment the nucleic acid sequence encoding for the target kinase comprises the sequence of SEQ ID NO: 29.

### Specific Embodiments

### EXAMPLE 1

### Plasmid of Kinase and Upstream Kinase (MAPK 14 and MKK 6)

HIS-tagged kinase and no-tag upstream kinase were designed for PCR production. PCR was conducted 30 cycles for MAPK 14 using primers 5′-CTTTAAGAAGGAGATATACCATGTCACAAGAAAGGCCTACATTCTACCGGCAGGA-3′ (SEQ ID NO: 35) and 5′-TGATGATGAGAACCCCCCCCGGACTCCATTTCTTCT-3′ (SEQ ID NO: 36) and MKK 6 using primers 5′-CTTTAAGAAGGAGATATACCATGTCACAATCAAAAGGTAAAAAGCGAAACCCTGG-3′ (SEQ ID NO: 37) and 5′-TGATGATGAGAACCCCCCCCTTAGTCTCCAAGAATCAGT-3′ (SEQ ID NO: 38). These amplified sequence were cloned separately into pIVEX2.3d vectors (Roche Diagnostics Corporation, Indianapolis, Ind., USA) and the sequence confirmed. A stop codon was engineered into the MKK6 sequence immediately following the last wild-type amino acid to prevent the addition of the hexa-histidine tag from being added.

### EXAMPLE 2

### Eukaryotic Cell Extract (HEK 293 Cell Extract)

Cells were treated with regulators for the desired time. Cells were harvested by removing media and rinsing cells once with ice-cold PBS. The PBS was removed and 0.5 ml cell lysis buffer added (20 mM Tris, pH 7.5, 150 mM NaCl, 1 mM EDTA, 1 mM EGTA, 1% TRITON X-100, 2.5 mM sodium pyrophosphate, 1 mM sodium vanadate Na₃VO₄, 1 μg/mL leupeptin, 1 mM PMSF) to T-flask and incubated on ice for 5 minutes. Cell debris was allowed to settle by gravity or gently centrifuged at approximately 4,000×g for 5 minutes. The supernatant was transferred to a new tube and the cell extract stored at −80° C.

### EXAMPLE 3

### Rapid Translation System (RTS) Reaction

The RTS components are commercially available from Roche Diagnostics Corporation, Indianapolis, Ind., USA. The reaction components were reconstituted per pack instructions as follows: E. coli extract, 0.525 ml reconstitution buffer (RB); reaction mixture, 0.25 ml RB; feeding mixture, 8.1 ml RB; amino acid mixture, 3 ml RB; and methionine, 1.8 ml RB. The RTS reaction (RTS 500) was set up as follows: the feeding solution contained 2.65 ml amino acid mixture, 0.3 ml methionine, and 8.1 ml feeding mixture; the reaction solution contained 0.525 ml E. coli extract, 0.225 ml reaction mixture, 0.27 ml amino acid mixture, and 0.03 ml methionine. Three separate reaction components were prepared. To each of the three reactions, 12 μg of MAPK 14 plasmid was added. An additional component, 100 μl HEK 293 cell extract, was added to the second reaction component, and an additional components, 100 μl HEK 293 cell extract and 5 μg MKK6 plasmid, were added to the third reaction component. The RTS reaction was run at 30° C. at 150 rpm for 24 hours in the RTS 500 Instrument.

### EXAMPLE 4

### Expression of Activated MAPK14 using Constitutively Active MKK6

A site-directed mutagenesis was performed on the MKK6 sequence (SEQ ID NO: 18) present in the pIVEX vector to alter serine 207 and threonine 211 to glutamic acid. Oligos were designed to introduce this mutation using the Site-Directed Mutagenesis Kit by Stratagene (La Jolla, Calif.). Forward primer 5′-CTTGGTGGACGAAGTTGCTAAAGAAATTGATGCAG-3′ (SEQ ID NO: 33) and reverse 5′-CTGCATCAATTTCTTTAGCAACTTCGTCCACCCAAG-3′ (SEQ ID NO: 34). The thermocycler profile was designed based on the pack insert. The sequence alteration was confirmed by DNA sequencing.

The RTS 500 components are commercially available from Roche Diagnostics Corporation, Indianapolis, Ind., USA. The reaction components were reconstituted per pack instructions as follows: E. coli extract, 0.525 ml reconstitution buffer (RB); reaction mixture, 0.25 ml RB; feeding mixture, 8.1 ml RB; amino acid mixture, 3 ml RB; and methionine, 1.8 ml RB. The RTS reaction (RTS 500) was set up as follows: the feeding solution contained 2.65 ml amino acid mixture, 0.3 ml methionine, and 8.1 ml feeding mixture; the reaction solution contained 0.525 ml E. coli extract, 0.225 ml reaction mixture, 0.27 ml amino acid mixture, and 0.03 ml methionine. To the reaction mix 25 μg MAPK14 plasmid and 2.5 μg constitutively active MKK6 (mMKK6; SEQ ID NO: 23) plasmid DNA was added. The RTS reaction was run at 30° C. at 150 rpm for 24 hours in the RTS 500 Instrument. Following purification detection of the activated form of MAPK14 was evaluated by detection using an anti-phospho p38 (i.e. MAPK14) antibody (Phospho-p38 MAPK Pathway Sampler Kit; Cell. Signaling Technology, Inc., Beverly, Mass., USA).

### EXAMPLE 4

### Ni-NTA Affinity Purification

The reaction mixture was removed from the RTS device (Rapid Translation System RTS 500 Instrument, Roche Diagnostics Corporation, Indianapolis, Ind., USA), and the soluble components and pellet were separated. The soluble portion of the protein was gently rocked with Ni-NTA resin at 4° C. for 1 hour in batch binding buffer (see below). The resin was packed into the column, and the column washed with washing buffer. The protein was then eluted with 500 mM imidazole, pH 8.0.

The binding buffer contained 20 mM Hepes, 300 mM NaCl, 2 mM MgCl₂, and 12 mM CHAPS, pH 8.0. The washing buffer contained 20 mM imidazole, 20 mM Hepes, 300 mM NaCl, 2 mM MgCl₂, and 12 mM CHAPS, pH 8.0. The elution buffer contained 500 mM imidazole, 20 mM Hepes, 500 mM NaCl, 2 mM MgCl₂, and 12 mM CHAPS, pH 8.0. Through further development experiments it was discovered that the best practice was to eliminate the CHAPS, but include 2 mM betametcaptoethanol as a reducing agent in the buffers.

### EXAMPLE 5

### Western Blot Detection of Active Kinase

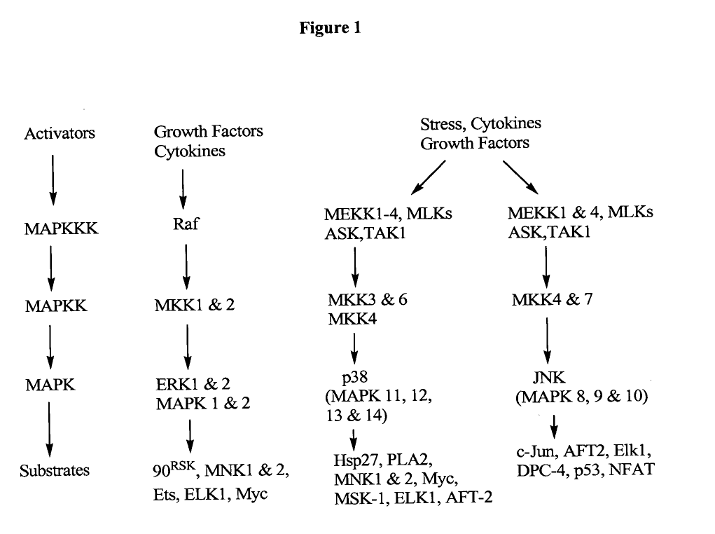

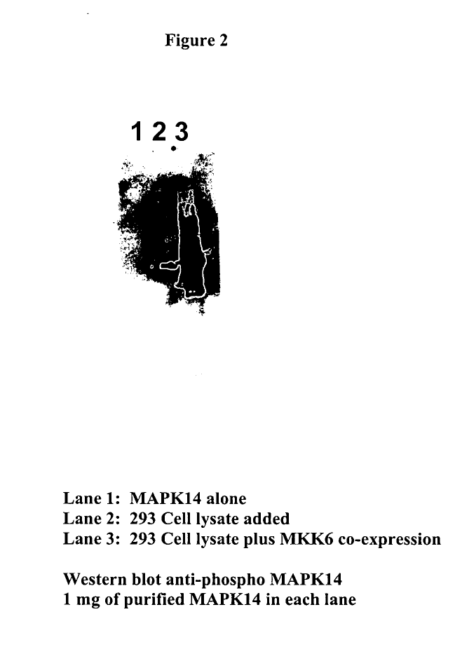

The purified protein was detected by anti-phospho p38 (i.e. MAPK14) antibody (Phospho-p38 MAPK Pathway Sampler Kit; Cell Signaling Technology, Inc., Beverly, Mass., USA). FIG. 2 shows the Western blot of 3 sets of RTS 500 reactions using an anti-phospho-p38 antibody for detection. Lanes 1-3 correspond to reaction sets 1-3, respectively, with reaction 1 being conducted in the absence of HEK 293 cell extract and MKK6 plasmid, reaction 2 being conducted in the absence of the MKK6 plasmid and reaction 3 being conducted in the presence of both the MAPK14 and MKK6 plasmids and the HEK 293 extract. As indicated in FIG. 1 co-expression of MAPK14 with MKK6 in the presence of an HEK 293 cell extract greatly enhances the phosphorylation of proteins present in the reaction.

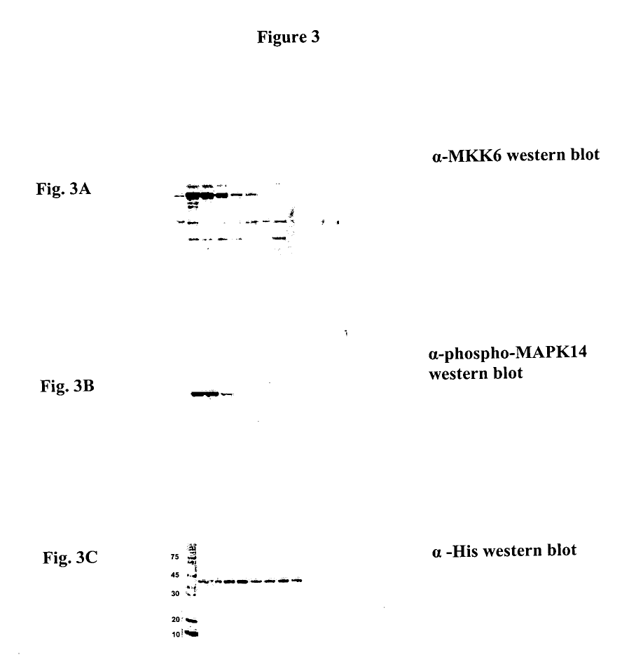

To further investigate the effect of the HEK 293 cell extract and MKK6 co-expression on the postranslational modification of MAPK14, the amount of phosphorylation was measured (using Western blot analysis with the p38 antibody) using different amount of HEK 293 cell extract or MKK6 plasmid. Applicants determined that MAPK14 expression levels were uneffected over a wide range of concentrations of eukaryotic cell extract. Furthermore, as shown in FIG. 3, when MAPK14 and MKK6 were co-expressed in the presence of a eukaryotic extract (HEK 293), as the concentration of the MKK6 plasmid present in the reaction was decreased, the amount of phosphorylation MAPK14 similarly decreased.

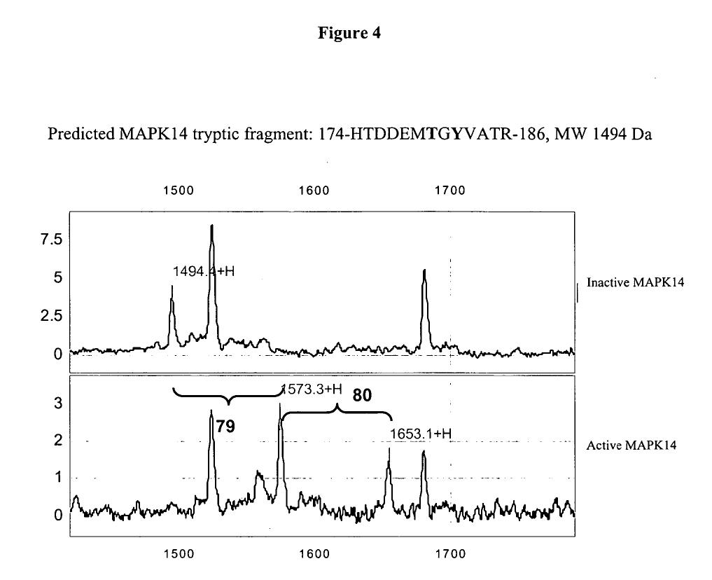

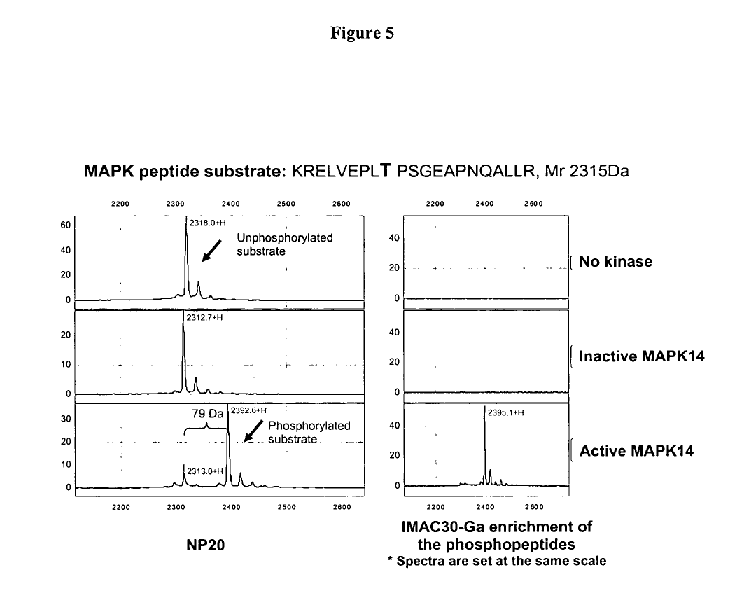

Mass spectrometry analysis was conducted on purified MAPK14 protein obtained from the in vitro expression reactions and an increase in mass of the intact MAPK14 protein from 41353 Da to 41170 Da was found for inactive and active MAPK14 proteins, respectively. This mass shift of approximately 183 Da is in the proximity of the mass of two phosphate residues (160 Da). Further analysis of tryptic fragment of 200 ng of the expressed MAPK14 protein (following a 60 minute trypsin digestion) revealed a shift in mass of the fragment 174-HTDDEMTGYVATR-186 SEQ ID NO: 20), from the predicted unmodified size of 1494 Da to fragments of 1573.3 and 1653.1. These two fragments represent the addition of one and two phosphate groups to the peptide fragment (see FIG. 4). Finally as shown in FIG. 5 kinase activity analysis reveals that only activated MAPK14 successfully phosphorylated the MAPK14 substrate KRELVEPLTPSGEAPNQALLR (SEQ ID NO: 21), shifting the molecular weight of the peptide from 2313 Da to 2392.6 Da.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

### EXAMPLE 5

### Activation of Other Kinases

Following the same method as described above, the following kinases have also been successfully activated: JNK 1 (MAPK8), JNK 2 (MAPK9), JNK 3 (MAPK10), MAPK 11, MAPK 12, MAPK 13, ERK3 (MAPK6), and ERK1 (MAPK3).

What is claimed is:

1. A method for expressing an activated target kinase in a cell free expression system, said method comprising
providing a reaction mixture comprising a prokaryotic cell extract, a eukaryotic cell extract, a gene encoding the target kinase, and a gene encoding an activating enzyme that activates said target protein, and
co-expressing the target kinase and the activating enzyme to produce the activated form of the target kinase.

2. The method of claim 1, further comprising the step of separating the activated target kinase from the mixture.

3. The method of claim 1 wherein the target kinase is selected from the group consisting of MAPK1, MAPK3, MAPK4, MAPK6, MAPK7, MAPK8, MAPK9, MAPK10, MAPK11, MAPK12, MAPK13 and MAPK14.

4. The method of claim 1 wherein the target kinase comprises an amino acid sequence selected from the group consisting of SEQ ID NO: 2, SEQ ID NO: 4, SEQ ID NO: 6, SEQ ID NO: 7, SEQ ID NO: 8, SEQ ID NO: 9, SEQ ID NO: 10, SEQ ID NO: 11 and SEQ ID NO: 12 and the activating enzyme comprises an amino acid sequence selected from the group consisting of SEQ ID NO; 15, SEQ ID NO: 16, SEQ ID NO: 18 and SEQ ID NO: 22.

5. The method of claim 4 wherein the activating enzyme comprises the amino acid sequence of SEQ ID NO: 18.

6. The method of claim 4 wherein the activating enzyme comprises the amino acid sequence of SEQ ID NO: 22.

7. The method of claim 1 wherein the target kinase comprises an amino acid sequence selected from the group consisting of SEQ ID NO: 6, SEQ ID NO: 7 and SEQ ID NO: 8, and the activating enzyme is selected from the group consisting of SEQ ID NO: 16, SEQ ID NO:18 and SEQ ID NO: 19.

8. The method of claim 1 wherein the target kinase is MAPK 14 and the enzyme is MKK6.

9. The method of claim 1 wherein the target kinase is selected from the group consisting of MAPK8, MAPK9, and MAPK10 and the activating enzyme is selected from the group consisting of MKK4, MKK6 and MKK7.

10. The method of claim 1 wherein the prokaryotic cell extract is an E. coli extract and the eukaryotic cell extract is an HEK 293 extract.

11. The method of claim 1 wherein the activated target kinase further comprises a peptide tag bound the carboxy terminus of the kinase via a linker.

12. The method of claim 11 wherein the linker is a labile linker.

13. The method of claim 1 wherein the gene encoding the target kinase comprises a nucleic acid sequence selected from the group consisting of SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32 and SEQ ID NO: 40, and the gene encoding the activating kinase comprises a nucleic acid sequence selected from the group consisting of SEQ ID NO: 23 and SEQ ID NO: 39.

14. A kit for the in vitro production of activated kinases, said kit comprising an expression reagent comprising a eukaryotic extract and a prokaryotic extract.

15. The kit of claim 14 further comprising a nucleic acid sequence encoding a target kinase selected from the group consisting of SEQ ID NO: 6, SEQ ID NO: 7, SEQ ID NO: 8, and SEQ ID NO: 12, and a nucleic acid sequence encoding an activating kinase selected from the group consisting of SEQ ID NO: 16, SEQ ID NO: 18 and SEQ ID NO: 19.

16. A kit for the in vitro production of activated kinases, said kit comprising
an expression reagent comprising a prokaryotic extract, a nucleic acid sequence encoding a target protein selected from the group consisting of SEQ ID NO: 6, SEQ ID NO: 7, SEQ ID NO: 8, and SEQ ID NO: 12, and a nucleic acid sequence encoding an activating protein comprising the sequence of SEQ ID NO: 22 or SEQ ID NO: 29.

17. The kit of claim 13 wherein the nucleic acid sequence encoding the target protein comprises the nucleic acid sequence of SEQ ID NO: 29 and the nucleic acid sequence encoding the activating protein comprises the sequence of SEQ ID NO: 23 or SEQ ID NO: 39.

18. A method for expressing an activated target kinase in an in vitro cell free expression system, said method comprising
providing a reaction mixture comprising a prokaryotic cell extract, a gene encoding the target kinase, and a gene encoding a constitutively active activating kinase;
co-expressing the target kinase and the constitutively active activating kinase to produce the activated form of the target kinase.

19. The method of claim 18 wherein the constitutively active activating kinase comprises the sequence of SEQ ID NO: 22.

20. The method of claim 19 wherein the activated target kinase further comprises a peptide tag bound to the carboxy terminus of the target kinase.


SEQUENCE LISTING (40 sequences)

SEQ ID NO: 1 - protein, 360 aa, Homo sapiens [sequence omitted]

SEQ ID NO: 2 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 3 - protein, 557 aa, Homo sapiens
MAEKGDCIASVYGYDLGGRFVDFQPLGFGVNGLVLSAVDSRACRKVAVKKIALSDARSMKHALREIKIIRRLDHDNIVKVYEVLGPKGTDLQGELFKFSVAYIVQEYMETDLARLLEQGTLAEEHAKLFMYQLLRGLKYIHSANVLHRDLKPANIFISTEDLVLKIGDFGLARIVDQHYSHKGYLSEGLVTKWYRSPRLLLSPNNYTKAIDMWAAGCILAEMLTGRMLFAGAHELEQMQLILETIPVIREEDKDELLRVMPSFVSSTWEVKRPLRKLLPEVNSEAIDFLEKILTFNPMDRLTAEMGLQHPYMSPYSCPEDEPTSQHPFRIEDEIDDIVLMAANQSQLSNWDTCSSRYPVSLSSDLEWRPDRCQDASEVQRDPRGFGALAEDVQVDPRKDSHSSSERFLEQSHSSMERAFEADYGRSCDYKVGSPSYLDKLLWRDNKPHHYSEPKLILDLSHWKQAAGAPPTATGLADTGAREDEPASLFLEIAQWVKSTQGAQSTPARPPTTPSAACLPRPPPPGPGGRRRQPPVRPGRVHLPRPEALHQARGPAGQ

SEQ ID NO: 4 - protein, 721 aa, Homo sapiens
MAEKFESLMNIHGFDLGSRYMDLKPLGCGGNGLVFSAVDNDCDKRVAIKKIVLTDPQSVKHALREIKIIRRLDHDNIVKVFEILGPSGSQLTDDVGSLTELNSVYIVQEYMETDLANVLEQGPLLEEHARLFMYQLLRGLKYIHSANVLHRDLKPANLFINTEDLVLKIGDFGLARIMDPHYSHKGHLSEGLVTKWYRSPRLLLSPNNYTKAIDMWAAGCIFAEMLTGKTLFAGAHELEQMQLILESIPVVHEEDRQELLSVIPVYIRNDMTEPHKPLTQLLPGISREALDFLEQILTFSPMDRLTAEEALSHPYMSIYSFPMDEPISSHPFHIEDEVDDILLMDETHSHIYNWERYHDCQFSEHDWPVHNNFDIDEVQLDPRALSDVTDEEEVQVDPRKYLDGDREKYLEDPAFDTNYSTEPCWQYSDHHENKYCDLECSHTCNYKTRSSSYLDNLVWRESEVNHYYEPKLIIDLSNWKEQSKEKSDKKGKSKCERNGLVKAQIALEEASQQLAGKEREKNQGFDFDSFIAGTIQLSSQHEPTDVVDKLNDLNSSVSQLELKSLISKSVSQEKQEKGMANLAQLEALYQSSWDSQFVSGGEDCFFINQFCEVRKDEQVEKENTYTSYLDKFFSRKEDTEMLETEPVEDGKLGERGHEEGFLNNSGEFLFNKQLESIGIPQFHSPVGSPLKSIQATLTPSAMKSSPQIPHQTYSSILKHLN

SEQ ID NO: 5 - protein, 815 aa, Homo sapiens
MAEPLKEEDGEDGSAEPPAREGRTRPHRCLCSAKNLALLKARSFDVTFDVGDEYEIIETIGNGAYGVVSSARRRLTGQQVAIKKIPNAFDVVTNAKRTLRELKILKHFKHDNIIAIKDILRPTVPYGEFKSVYVVLDLMESDLHQIIHSSQPLTLEHVRYFLYQLLRGLKYMHSAQVIHRDLKPSNLLVNENCELKIGDFGMARGLCTSPAEHQYFMTEYVATRWYRAPELMLSLHEYTQAIDLWSVGCIFGEMLARRQLFPGKNYVHQLQLIMMVLGTPSPAVIQAVGAERVRAYIQSLPPRQPVPWETVYPGADRQALSLLGRMLRFEPSARISAAAALRHPFLAKYHDPDDEPDCAPPFDFAFDREALTRERIKEAIVAEIEDFHARREGIRQQIRFQPSLQPVASEPGCPDVEMPSPWAPSGDCAMESPPPAPPPCPGPAPDTIDLTLQPPPPVSEPAPPKKDGAISDNTKAALKAALLKSLRSRLRDGPSAPLEAPEPRKPVTAQERQREREEKRRRRQERAKEREKRRQERERKERGAGASGGPSTDPLAGLVLSDNDRSLLERWTRMARPAAPALTSVPAPAPAPTPTPTPVQPTSPPPGPLAQPTGPQPQSAGSTSGPVPQPACPPPGPAPHPTGPPGPIPVPAPPQIATSTSLLAAQSLVPPPGLPGSSTPGVLPYFPPGLPPPDAGGAPQSSMSESPDVNLVTQQLSKSQVEDPLPPVFSGTPKGSGAGYGVGFDLEEFLNQSFDMGVADGPQDGQADSASLSASLLADWLEGHGMNPADIESLQREIQMDSPMLLADLPDLQDP

SEQ ID NO: 6 - protein, 427 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 7 - protein, 424 aa, Homo sapiens [sequence omitted]

SEQ ID NO: 8 - protein, 464 aa, Homo sapiens
MSLHFLYYCSEPTLDVKIAFCQGFDKQVDVSYIAKHYNMSKSKVDNQFYSVEVGDSTFTVLKRYQNLKPIGSGAQGIVCAAYDAVLDRNVAIKKLSRPFQNQTHAKRAYRELVLMKCVNHKNIISLLNVFTPQKTLEEFQDVYLVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMVRHKILFPGRDYIDQWNKVIEQLGTPCPEFMKKLQPTVRNYVENRPKYAGLTFPKLFPDSLFPADSEHNKLKASQARDLLSKMLVIDPAKRISVDDALQHPYINVWYDPAEVEAPPPQIYDKQLDEREHTIEEWKELIYKEVMNSEEKTKNGVVKGQPSPSGAAVNSSESLPPSSSVNDISSMSTDQTLASDTDSSLEASAGPLGCCR

SEQ ID NO: 9 - protein, 372 aa, Homo sapiens
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQAGAHQGARLALDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ

SEQ ID NO: 10 - protein, 367 aa, Homo sapiens
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQADSEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 11 - protein, 365 aa, Homo sapiens
MSLIRKKGFYKQDVNKTAWELPKTYVSPTHVGSGAYGSVCSAIDKRSGEKVAIKKLSRPFQSEIFAKRAYRELLLLKHMQHENVIGLLDVFTPASSLRNFYDFYLVMPFMQTDLQKIMGMEFSEEKIQYLVYQMLKGLKYIHSAGVVHRDLKPGNLAVNEDCELKILDFGLARHADAEMTGYVVTRWYRAPEVILSWMHYNQTVDIWSVGCIMAEMLTGKTLFKGKDYLDQLTQILKVTGVPGTEFVQKLNDKAAKSYIQSLPQTPRKDFTQLFPRASPQAADLLEKMLELDVDKRLTAAQALTHPFFEPFRDPEEETEAQQPFDDSLEHEKLTVDEWKQHIYKEIVNFSPIARKDSRRRSGMKL

SEQ ID NO: 12 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 13 - protein, 392 aa, Homo sapiens
PKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDAAETPPRPRTPGRPLSSYGMDSRPPMAIFELLDYIVNEPPPKLPSGVFSLEFQDFVNKCLIKNPAERADLKQLMVHAFIKRSDAEEVDFAGWLCSTIGLNQPSTPTHAAGV

SEQ ID NO: 14 - protein, 400 aa, Homo sapiens [sequence omitted]

SEQ ID NO: 15 - protein, 347 aa, Homo sapiens
MESPASSQPASMPQSKGKSKRKKDLRISCMSKPPAPNPTPPRNLDSRTFITIGDRNFEVEADDLVTISELGRGAYGVVEKVRHAQSGTIMAVKRIRATVNSQEQKRLLMDLDINMRTVDCFYTVTFYGALFREGDVWICMELMDTSLDKFYRKVLDKNMTIPEDILGEIAVSIVRALEHLHSKLSVIHRDVKPSNVLINKEGHVKMCDFGISGYLVDSVAKTMDAGCKPYMAPERINPELNQKGYNVKSDVWSLGITMIEMAILRFPYESWGTPFQQLKQVVEEPSPQLPADRFSPEFVDFTAQCLRKNPAERMSYLELMEHPFFTLHKTKKTDIAAFVKEILGEDS

SEQ ID NO: 16 - protein, 399 aa, Homo sapiens
MAAPSPSGGGGSGGGSGSGTPGPVGSPAPGHPAVSSMQGKRKALKLNFANPPFKSTARFTLNPNPTGVQNPHIERLRTHSIESSGKLKISPEQHWDFTAEDLKDLGEIGRGAYGSVNKMVHKPSGQIMAVKRIRSTVDEKEQKQLLMDLDVVMRSSDCPYIVQFYGALFREGDCWICMELMSTSFDKFYKYVYSVLDDVIPEEILGKITLATVKALNHLKENLKIIHRDIKPSNILLDRSGNIKLCDFGISGQLVDSIAKTRDAGCRPYMAPERIDPSASRQGYDVRSDVWSLGITLYELATGRFPYPKWNSVFDQLTQVVKGDPPQLSNSEEREFSPSFINFVNLCLTKDESKRPKYKELLKHPFILMYEERAVEVACYVCKILDQMPATPSSPMYVD

SEQ ID NO: 17 - protein, 438 aa, Homo sapiens
MLWLALGPFPAMENQVLVIRIKIPNSGAVDWTVHSGPQLLFRDVLDVIGQVLPEATTTAFEYEDEDGDRITVRSDEEMKAMLSYYYSTVMEQQVNGQLIEPLQIFPRACKPPGERNIHGLKVNTRAGPSQHSSPAVSDSLPSNSLKKSSAELKKILANGQMNEQDIRYRDTLGHGNGGTVYKAYHVPSGKILAVKVILLDITLELQKQIMSELEILYKCDSSYIIGFYGAFFVENRISICTEFMDGGSLDVYRKMPEHVLGRIAVAVVKGLTYLWSLKILHRDVKPSNMLVNTRGQVKLCDFGVSTQLVNSIAKTYVGTNAYMAPERISGEQYGIHSDVWSLGISFMEIQKNQGSLMPLQLLQCIVDEDSPVLPVGEFSEPFVHFITQCMRKQPKERPAPEELMGHPFIVQFNDGNAAVVSMWVCRALEERRSQQGPP

SEQ ID NO: 18 - protein, 334 aa, Homo sapiens
MSQSKGKKRNPGLKIPKEAFEQPQTSSTPPRDLDSKACISIGNQNFEVKADDLEPIMELGRGAYGVVEKMRHVPSGQIMAVKRIRATVNSQEQKRLLMDLDISMRTVDCPFTVTFYGALFREGDVWICMELMDTSLDKFYKQVIDKGQTIPEDILGKIAVSIVKALEHLHSKLSVIHRDVKPSNVLINALGQVKMCDFGISGYLVDSVAKTIDAGCKPYMAPERINPELNQKGYSVKSDIWSLGITMIELAILRFPYDSWGTPFQQLKQVVEEPSPQLPADKFSAEFVDFTSQCLKKNSKERPTYPELMQHPFFTLHESKGTDVASFVKLILGD

SEQ ID NO: 19 - protein, 418 aa, Homo sapiens
MAASSLEQKLSRLEAKLKQENREARRRIDLNLDISPQRPRPTLQLPLANDGGSRSPSSESSPQHPTPPARPRHMLGLPSTLFTPRSMESIEIDQKLQEIMKQTGYLTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDSKAKTRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDMAKTESPRTSGVLSQPHLPFFR

SEQ ID NO: 20 - protein, 13 aa, Homo sapiens
HTDDEMTGYVATR

SEQ ID NO: 21 - protein, 21 aa, Homo sapiens
KRELVEPLTPSGEAPNQALLR

SEQ ID NO: 22 - protein, 334 aa, Artificial Sequence
  muntant amino acid sequence of consitutive active MKK6
  MISC_FEATURE (207)..(207): serine residue at position 207 of the wild type sequence has been replaced with glutamic acid
  MISC_FEATURE (211)..(211): threonine residue at position 211 of the wild type sequence is replaced with glutamic acid.
MSQSKGKKRNPGLKIPKEAFEQPQTSSTPPRDLDSKACISIGNQNFEVKADDLEPIMELGRGAYGVVEKMRHVPSGQIMAVKRIRATVNSQEQKRLLMDLDISMRTVDCPFTVTFYGALFREGDVWICMELMDTSLDKFYKQVIDKGQTIPEDILGKIAVSIVKALEHLHSKLSVIHRDVKPSNVLINALGQVKMCDFGISGYLVDEVAKEIDAGCKPYMAPERINPELNQKGYSVKSDIWSLGITMIELAILRFPYDSWGTPFQQLKQVVEEPSPQLPADKFSAEFVDFTSQCLKKNSKERPTYPELMQHPFFTLHESKGTDVASFVKLILGD

SEQ ID NO: 23 - DNA, 1005 bp, Homo sapiens
atgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagacttg

SEQ ID NO: 24 - DNA, 4193 bp, Homo sapiens
catggcggcgactgcggcaaagcgagagcctcggagacgccgctgccgccagcacagccggagatctgagccgacactgggggcagtccgcgagccccgcactctctcgatgagtcggagaagtcccgttgtatcagagtaagatggacggtagctttgattgtgattgtggtgagctggagccacctgatcactaacaaaagacatcttctgttaaccaacagccgccaggcttcctgttgaaataaatatatagcaacaaaggaaaaaaagaagcaaaacggaaatagtgcttaccagcaccttagaatgatgctgctcaggaccagtccaacactgaatgtatctgcactgtgaggagaatgttcatagaagcctgttgtgtgcatatttattcactttttgttaaatgttaaatcgtttagcacggtaatctgagtgcacagtatgtcatttcattccgtttgagtttcttgttttcgttaaatgtctgcagagttgctgcccctttcttgaactatgagtactgcaatctttttaattctcaatatgaatagagctttttgagctttaaatctaaggggaactcgacaggcctgtttggcatatgcaatgaacatcaagaaaccatcttgctgtggaagcataattatttttcttctccctttttgaaagatctttccttttgatgccagttttcttccttgtttacacaagttcaatttgaaaggaaaaggcaatagtaagggtttcaaaatggcagagaaatttgaaagtctcatgaacattcatggttttgatctgggttctaggtatatggacttaaaaccattgggttgtggaggcaatggcttggttttttctgctgtagacaatgactgtgacaaaagagtagccatcaagaaaattgtccttactgatccccagagtgtcaaacatgctctacgtgaaatcaaaattattagaagacttgaccatgataacattgtgaaagtgtttgagattcttggtcccagtggaagccaattaacagacgatgtgggctctcttacggaactgaacagtgtttacattgttcaggagtacatggagacagacttggctaatgtgctggagcagggccctttactggaagagcatgccaggcttttcatgtatcagctgctacgggggctcaagtatattcactctgcaaatgtactgcacagagatctcaaaccagctaatcttttcattaatacggaagacttggtgctgaagataggtgactttggtcttgcacggatcatggatcctcattattcccataagggtcatctttctgaaggattggttactaaatggtacagatctccacgtcttttactttctcctaataattatactaaagccattgacatgtgggctgcaggctgcatctttgctgaaatgctgactggtaaaaccctttttgcaggtgcacatgaacttgaacagatgcagctgattttagaatctattcctgttgtacatgaggaagatcgtcaggagcttctcagcgtaattccagtttacattagaaatgacatgactgagccacacaaacctttaactcagctgcttccaggaattagtcgagaagcactggatttcctggaacaaattttgacatttagccccatggatcggttaacagcagaagaagcactctcccatccttacatgagcatatattcttttccaatggatgagccaatttcaagccatccttttcatattgaagatgaagttgatgatattttgcttatggatgaaactcacagtcacatttataactgggaaaggtatcatgattgtcagttttcagagcatgattggcctgtacataacaactttgatattgatgaagttcagcttgatccaagagctctgtccgatgtcactgatgaagaagaagtacaagttgatccccgaaaatatttggatggagatcgggaaaagtatctggaggatcctgcttttgataccaattactctactgagccttgttggcaatactcagatcatcatgaaaacaaatattgtgatctggagtgtagccatacttgtaactacaaaacgaggtcatcatcatatttagataacttagtttggagagagagtgaagttaaccattactatgaacccaagcttattatagatctttccaattggaaagaacaaagcaaagaaaaatctgataagaaaggcaaatcaaaatgtgaaaggaatggattggttaaagcccagatagcgctagaggaagcatcacagcaactggctggaaaagaaagggaaaagaatcagggatttgattttgattcctttattgcaggaactattcagcttagttcccagcatgagcctactgatgttgttgataaattaaatgacttgaatagctcagtgtcccaactagaattgaaaagtttgatatcaaagtcagtaagccaagaaaaacaggaaaaaggaatggcaaatctggctcaattagaagccttgtaccagtcttcttgggacagccagtttgtgagtggtggggaggactgttttttcataaatcagttttgtgaggtaaggaaggatgaacaagttgagaaggaaaacacttacactagttacttggacaagttctttagcaggaaagaagatactgaaatgctagaaactgagccagtagaggatgggaagcttggggagagaggacatgaggaaggatttctgaacaacagtggggagttcctctttaacaagcagctcgagtccataggcatcccacagtttcacagtccagttgggtcaccacttaagtcaatacaggccacattaacaccttctgctatgaaatcttcccctcaaattcctcatcaaacatacagcagcattctgaaacatctgaactaaaacactcagcagacatttatctttgtattcttcatgaaatgtgttttgtctttttttattactagtgtttaagtcattttttacttgaatcagatggtgtcatttagtaaggattttatgagttcttgttttttaaaatccagactttctttttctacatgtgagatagttttcattttaactggcatgtcatttgcacacaaaaataaagactagagcaaaataatgcaacgcaggaggagaaaagaaatgcactaagacaagaacattctctcatagaacattgatctgttttacaggaaacaaaccttgccttgaaatttacacagtgagactgtacataattgcatgaaaatagctatttttttcctaagacatttttcattcatgaatattttcaagtttttcatactgtacacatttcttaaaacacatgataccagcagcaactgaaaatgaatgccgaatttggtacacatgtgttatctacctcaaggtaacaagagtatgtggcaaaacatataccacccatagtgcttcacaaaatgcacttctatttagccagcgtttattgtagtaaactattcttaataaaactcactcactgtttataaatgttctggtatgcattctttatagtgaagtgttaatacatcacatcttatttattttagcaaatcagtatattttctgtatttaattataaaaaattaacttagtttttaaaatttatttgcaaatatactttttccatttggcactatggtttgttgcctacctagctgcatctataatgtcagcttatcctaaggctgtccacgtacttaatttacttaagtgttcattttaagtaacgtgctcactgtgtataggaatttgtattttggaggtgcttgatctatctacaaagaaaaattaattaggaattactttattataaaatgctcctagaagtcttaattgtgtttattttttaaaaaaacaaatgttagacttgtgtgcatggaagtaattaaggtacatcattattgtagtttgaaagttgtacatgataagacattttgtttttactgtatgtttttactgaatgatctattccccatcccaaggcaagcatgaataaaattaggttaaacgtagcatgtggcatcgcagtctcttagaatttgtttcatctattttattttattgaatactgtctgtatctttggttatcctgtttgaagaaaaaggacaaataaaacatggccagcaaatacaaaaaaaaaaaa

SEQ ID NO: 25 - DNA, 2828 bp, Homo sapiens
gaattccggagacccccgcgctggggacgggaggccggcgagcctcgggacctctgaaagccttgaggaggcccggggacaccatggccgagcctctgaaggaggaagacggcgaggacggctctgcggagcccccggcccgtgaaggtcgaacccgcccacaccgctgcctctgtagcgccaagaacctggccctgcttaaagcccgctccttcgatgtgacctttgacgtgggcgacgagtacgagatcatcgagaccataggcaacggggcctatggagtggtgtcctccgcccgccgccgcctcaccggccagcaggtggccatcaagaagatccctaatgctttcgatgtggtgaccaatgccaagcggaccctcagggagctgaagatcctcaagcactttaaacacgacaacatcatcgccatcaaggacatcctgaggcccaccgtgccctatggcgaattcaaatctgtctacgtggtcctggacctgatggaaagcgacctgcaccagatcatccactcctcacagcccctcacactggaacacgtgcgctacttcctgtaccaactgctgcggggcctgaagtacatgcactcggctcaggtcatccaccgtgacctgaagccctccaacctattggtgaatgagaactgtgagctcaagattggtgactttggtatggctcgtggcctgtgcacctcgcccgctgaacatcagtacttcatgactgagtatgtggccacgcgctggtaccgtgcgcccgagctcatgctctctttgcatgagtatacacaggctattgacctctggtctgtgggctgcatctttggtgagatgctggcccggcgccagctcttcccaggcaaaaactatgtacaccagctacagctcatcatgatggtgctgggtaccccatcaccagccgtgattcaggctgtgggggctgagagggtgcgggcctatatccagagcttgccaccacgccagcctgtgccctgggagacagtgtacccaggtgccgaccgccaggccctatcactgctgggtcgcatgctgcgttttgagcccagcgctcgcatctcagcagctgctgcccttcgccaccctttcctggccaagtaccatgatcctgatgatgagcctgactgtgccccgccctttgactttgcctttgaccgcgaagccctcactcgggagcgcattaaggaggccattgtggctgaaattgaggacttccatgcaaggcgtgagggcatccgccaacagatccgcttccagccttctctacagcctgtggctagtgagcctggctgtccagatgttgaaatgcccagtccctgggctcccagtggggactgtgccatggagtctccaccaccagccccgccaccatgccccggccctgcacctgacaccattgatctgaccctgcagccacctccaccagtcagtgagcctgccccaccaaagaaagatggtgccatctcagacaatactaaggctgcccttaaagctgccctgctcaagtctttgaggagccggctcagagatggccccagcgcacccctggaggctcctgagcctcggaagccggtgacagcccaggagcgccagcgggagcgggaggagaagcggcggaggcggcaagaacgagccaaggagcgggagaaacggcggcaggagcgggagcgaaaggaacggggggctggggcctctgggggcccctccactgaccccttggctggactagtgctcagtgacaatgacagaagcctgttggaacgctggactcgaatggcccggcccgcagccccagccctcacctctgtgccggcccctgccccagcgccaacgccaaccccaaccccagtccaacctaccagtcctcctcctggccctctagcccagcccactggcccgcaaccacaatctgcgggctctacctctggccctgtaccccagcctgcctgcccaccccctggccctgcaccccaccccactggccctcctgggcccatccctgtccccgcgccaccccagattgccacctccaccagcctcctggctgcccagtcacttgtgccaccccctgggctgcctggctccagcaccccaggagttttgccttacttcccacctggcctgccgcccccagacgccgggggagcccctcagtcttccatgtcagagtcacctgatgtcaaccttgtgacccagcagctatctaagtcacaggtggaggaccccctgccccctgtgttctcaggcacaccaaagggcagtggggctggctacggtgttggctttgacctggaggaattcttaaaccagtctttcgacatgggcgtggctgatgggccacaggatggccaggcagattcagcctctctctcagcctccctgcttgctgactggctcgaaggccatggcatgaaccctgccgatattgagtccctgcagcgtgagatccagatggactccccaatgctgctggctgacctgcctgacctccaggacccctgaggcccccagcctgtgccttgctgccacagtagacctagttccaggatccatgggagcattctcaaaggctttagccctggacccagcaggtgaggctcggcttggattattctgcaggttcatctcagacccacctttcagccttaagcagccacctgagccaccaccgagccatggcaggatcgggagaccccaactccccctgaacaatccttttcagtattatatttttattattattatgttattattacactgtcttttgccatcaaaatgaggcctgtgaaatacaaggtt

SEQ ID NO: 26 - DNA, 1418 bp, Homo sapiens
cattaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgtggatttatggtctgtggggtgcattatgggagaaatggtttgccacaaaatcctctttccaggaagggactatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttagcacaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgta

SEQ ID NO: 27 - DNA, 1873 bp, Homo sapiens
caaactacgtgctgtacagctgcatcagctgctcgtagacatgtccagcagctggtcgaggtccacgccgcggtaggtgaagttgcggaaggtccggcgagggatctgaaacttgccccttacccttcgggatattgcaggacgctgcatcatgagcgacagtaaatgtgacagtcagttttatagtgtccaagtggcagactcaaccttcactgtcctaaaacgttaccagcagctgaaaccaattggctctggggcccaagggattgtttgtgctgcatttgatacagttcttgggataaatgttgcagtcaagaaactaagccgtccttttcagaaccaaactcatgcaaagagagcttatcgtgaacttgtcctcttaaaatgtgtcaatcataaaaatataattagtttgttaaatgtgtttacaccacaaaaaactctagaagaatttcaagatgtgtatttggttatggaattaatggatgctaacttatgtcaggttattcacatggagctggatcatgaaagaatgtcctaccttctttaccagatgctttgtggtattaaacatctgcattcagctggtataattcatagagatttgaagcctagcaacattgttgtgaaatcagactgcaccctgaagatccttgactttggcctggcccggacagcgtgcactaacttcatgatgaccccttacgtggtgacacggtactaccgggcgcccgaagtcatcctgggtatgggctacaaagagaacgttgatatctggtcagtgggttgcatcatgggagagctggtgaaaggttgtgtgatattccaaggcactgaccatattgatcagtggaataaagttattgagcagctgggaacaccatcagcagagttcatgaagaaacttcagccaactgtgaggaattatgtcgaaaacagaccaaagtatcctggaatcaaatttgaagaactctttccagattggatattcccatcagaatctgagcgagacaaaataaaaacaagtcaagccagagatctgttatcaaaaatgttagtgattgatcctgacaagcggatctctgtagacgaagctctgcgtcacccatacatcactgtttggtatgaccccgccgaagcagaagccccaccacctcaaatttatgatgcccagttggaagaaagagaacatgcaattgaagaatggaaagagctaatttacaaagaagtcatggattgggaagaaagaagcaagaatggtgttgtaaaagatcagcctccagatgcagcagtaagtagcaacgccactccttctcagtcttcatcgatcaatgacatttcatccatgtccactgagcagacgctggcctcagacacagacagcagtcttgatgcctcgacgggaccccttgaaggctgtcgatgataggttagaaatagcaaacctgtcagcattgaaggaactctcacctccgtgggcctgaaatgcttgggagttgatggaaccaaatagaaaaactccatgttctgcatgtaagaaacacaatgccttgccctactcagacctgataggattgcctgcttagatgataaaatgaggcagaatatgtctgaagaaaaaaattgcaagccacacttctagagattttgttcaagatcatttcagttgagcagttagagtaggtgaatttgtcaaattgtactagtgacagtttctcatcatctgtaactgttgagatgattgtgcatgtgaccacaaatgcttgcttggacttgcccatctagcactttggaaatcagtatttaaatgccaaataatcttccaggtagtgctgcttctgaagttatctcttaatcctcttaagtaatttgg

SEQ ID NO: 28 - DNA, 2211 bp, Homo sapiens
ttatgcaagaaactgttgaattagacccgtttcctatagatgagaaaccatacaagctgtggtatttatgagcctccatttcttatactactgcagtgaaccaacattggatgtgaaaattgccttttgtcagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacattattagtttattaaatgtcttcacaccccagaaaacgctggaggagttccaagatgtttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctacaaggagaacgtggatatatggtctgtgggatgcattatgggagaaatggttcgccacaaaatcctctttccaggaagggactatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcaggtgcagcagtgaacagcagtgagagtctccctccatcctcgtctgtcaatgacatctcctccatgtccaccgaccagaccctggcatctgacactgacagcagcctggaagcctcggcaggacccctgggttgttgcaggtgactagccgcctgcctgcgaaacccagcgttcttcaggagatgatgtgatggaacacacacacacgcagacacacacacacacacaaatgcagacacacaacatcaagaaaacagcaagggagagaatccaagcctaaaattaaataaatctttcagcctgcttcttccccagggttctgtattgcagctaagctcaaatgtatatttaacttctagttgctcttgctttggtcttcttccaatgatgcttactacagaaagcaaatcagacacaattagagaagccttttccataaagtgtaattttaatggctgcaaaaccggcaacctgtaactgcccttttaaatggcatgacaaggtgtgcagtggccccatccagcatgtgtgtgtctctatcttgcatctacctgctccttggcctagtcagatggatgtagatacagatccgcatgtgtctgtattcatacagcactacttacttagagatgctactgtcagtgtcctcagggctctaccaagacataatgcactggggtaccacatggtccatttcatgtgatctattactctgacataaacccatctgtaatatattgccagtatataagctgtttagtttgttaattgattaaactgtatgtcttataagaaaacatgtaaagggggaatatatggggggagtgagctctctcagacccttgaagatgtagcttccaaatttgaatggattaaatggcacctgtatacca

SEQ ID NO: 29 - DNA, 1083 bp, Homo sapiens
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtccttg

SEQ ID NO: 30 - DNA, 2180 bp, Homo sapiens
gtgaaattctgctccggacatgtcgggccctcgcgccggcttctaccggcaggagctgaacaagaccgtgtgggaggtgccgcagcggctgcaggggctgcgcccggtgggctccggcgcctacggctccgtctgttcggcctacgacgcccggctgcgccagaaggtggcggtgaagaagctgtcgcgccccttccagtcgctgatccacgcgcgcagaacgtaccgggagctgcggctgctcaagcacctgaagcacgagaacgtcatcgggcttctggacgtcttcacgccggccacgtccatcgaggacttcagcgaagtgtacttggtgaccaccctgatgggcgccgacctgaacaacatcgtcaagtgccaggcgggcgcccatcagggtgcccgcctggcacttgacgagcacgttcaattcctggtttaccagctgctgcgcgggctgaagtacatccactcggccgggatcatccaccgggacctgaagcccagcaacgtggctgtgaacgaggactgtgagctcaggatcctggatttcgggctggcgcgccaggcggacgaggagatgaccggctatgtggccacgcgctggtaccgggcacctgagatcatgctcaactggatgcattacaaccaaacagtggatatctggtccgtgggctgcatcatggctgagctgctccagggcaaggccctcttcccgggaagcgactacattgaccagctgaagcgcatcatggaagtggtgggcacacccagccctgaggttctggcaaaaatctcctcggaacacgcccggacatatatccagtccctgccccccatgccccagaaggacctgagcagcatcttccgtggagccaaccccctggccatagacctccttggaaggatgctggtgctggacagtgaccagagggtcagtgcagctgaggcactggcccacgcctacttcagccagtaccacgaccccgaggatgagccagaggccgagccatatgatgagagcgttgaggccaaggagcgcacgctggaggagtggaaggagctcacttaccaggaagtccttagcttcaagcccccagagccaccgaagccacctggcagcctggagattgagcagtgaggtgctgcccagcagcccctgagagcctgtggaggggcttgggcctgcacccttccacagctggcctggtttcctcgagaggcacctcccacactcctatggtcacagacttctggcctaggacccctcgccttcaggagaatctacacgcatgtatgcatgcacaaacatgtgtgtacatgtgcttgccatgtgtaggagtctgggcacaagtgtccctgggcctaccttggtcctcctgtcctcttctggctactgcactctccactgggacctgactgtggggtcctagatgccaaaggggttcccctgcggagttcccctgtctgtcccaggccgacccaagggagtgtcagccttgggctctcttctgtcccagggctttctggagggcgcgctggggccgggaccccgggagactcaaagggagaggtctcagtggttagagctgctcagcctggaggtagggcgctgtcttggtcactgctgagacccacaggtctaagaggagaggcagagccagtgtgccaccaggctgggcagggacaaccaccaggtgtcaaatgagaaaagctgcctggagtcttgtgttcacccgtgggtgtgtgtgggcacgtgtggatgagcgtgcactccccgtgttcatatgtcagggcacatgtgatgtggtgcgtgtgaatctgtgggcgcccaaggccagcagccatatctggcaagaagctggagccggggtgggtgtgctgttgccttccctctcctcggttcctgatgccttgaggggtgtttcagactggcggcaccgttgtggccctgcagccggagatctgaggtgctctggtctgtgggtcagtcctctttccttgtcccaggatggagctgatccagtaacctcggagacgggaccctgcccagagctgagttgggggtgtggctctgccctggaaagggggtgacctcttgcctcgaggggcccagggaagcctgggtgtcaagtgcctgcaccaggggtgcacaataaagggggttctctctcagaaaaaaa

SEQ ID NO: 31 - DNA, 1104 bp, Homo sapiens
atgagctctccgccgcccgcccgcagtggcttttaccgccaggaggtgaccaagacggcctgggaggtgcgcgccgtgtaccgggacctgcagcccgtgggctcgggcgcctacggcgcggtgtgctcggccgtggacggccgcaccggcgctaaggtggccatcaagaagctgtatcggcccttccagtccgagctgttcgccaagcgcgcctaccgcgagctgcgcctgctcaagcacatgcgccacgagaacgtgatcgggctgctggacgtattcactcctgatgagaccctggatgacttcacggacttttacctggtgatgccgttcatgggcaccgacctgggcaagctcatgaaacatgagaagctaggcgaggaccggatccagttcctcgtgtaccagatgctgaaggggctgaggtatatccacgctgccggcatcatccacagagacctgaagcccggcaacctggctgtgaacgaagactgtgagctgaagatcctggacttcggcctggccaggcaggcagacagtgagatgactgggtacgtggtgacccggtggtaccgggctcccgaggtcatcttgaattggatgcgctacacgcagacggtggacatctggtccgtgggctgcatcatggcggagatgatcacaggcaagacgctgttcaagggcagcgaccacctggaccagctgaaggagatcatgaaggtgacggggacgcctccggctgagtttgtgcagcggctgcagagcgatgaggccaagaactacatgaagggcctccccgaattggagaagaaggattttgcctctatcctgaccaatgcaagccctctggctgtgaacctcctggagaagatgctggtgctggacgcggagcagcgggtgacggcaggcgaggcgctggcccatccctacttcgagtccctgcacgacacggaagatgagccccaggtccagaagtatgatgactcctttgacgacgttgaccgcacactggatgaatggaagcgtgttacttacaaagaggtgctcagcttcaagcctccccggcagctgggggccagggtctccaaggagacacctctgttg

SEQ ID NO: 32 - DNA, 1888 bp, Homo sapiens
gcggcgcggggcgggcgcagcgggggtcggggcgctgggagcccgttgggccgcgaacgcagccgccacgccggggccgccgagatcgggtgcccgggatgagcctcatccggaaaaagggcttctacaagcaggacgtcaacaagaccgcctgggagctgcccaagacctacgtgtccccgacgcacgtcggcagcggggcctatggctccgtgtgctcggccatcgacaagcggtcaggggagaaggtggccatcaagaagctgagccgaccctttcagtccgagatcttcgccaagcgcgcctaccgggagctgctgctgctgaagcacatgcagcatgagaacgtcattgggctcctggatgtcttcaccccagcctcctccctgcgcaacttctatgacttctacctggtgatgcccttcatgcagacggatctgcagaagatcatggggatggagttcagtgaggagaagatccagtacctggtgtatcagatgctcaaaggccttaagtacatccactctgctggggtcgtgcacagggacctgaagccaggcaacctggctgtgaatgaggactgtgaactgaagattctggattttgggctggcgcgacatgcagacgccgagatgactggctacgtggtgacccgctggtaccgagcccccgaggtgatcctcagctggatgcactacaaccagacagtggacatctggtctgtgggctgtatcatggcagagatgctgacagggaaaactctgttcaaggggaaagattacctggaccagctgacccagatcctgaaagtgaccggggtgcctggcacggagtttgtgcagaagctgaacgacaaagcggccaaatcctacatccagtccctgccacagacccccaggaaggatttcactcagctgttcccacgggccagcccccaggctgcggacctgctggagaagatgctggagctagacgtggacaagcgcctgacggccgcgcaggccctcacccatcccttctttgaacccttccgggaccctgaggaagagacggaggcccagcagccgtttgatgattccttagaacacgagaaactcacagtggatgaatggaagcagcacatctacaaggagattgtgaacttcagccccattgcccggaaggactcacggcgccggagtggcatgaagctgtagggactcatcttgcatggcaccaccggccagacactgcccaaggaccagtatttgtcactaccaaactcagcccttcttggaatacagcctttcaagcagaggacagaagggtccttctccttatgtgggaaatgggcctagtagatgcagaattcaaagatgtcggttgggagaaactagctctgatcctaacaggccacgttaaactgcccatctggagaatcgcctgcaggtggggccctttccttcccgccagagtggggctgagtgggcgctgagccaggccgggggcctatggcagtgatgctgtgttggtttcctagggatgctctaacgaattaccacaaacctggtggattgaaacagcagaacttgattcccttacagttctggaggctggaaatctgggatggaggtgttggcagggctgtggtccctttgaaggctctggggaagaatccttccttggctctttttagcttgtggcggcagtgggcagtccgtggcattccccagcttattgctgcatcactccagtctctgtctcttctgttctctcctcttttaacaacagtcattggatttagggcccaccctaatcctgtgtgatcttatcttgatccttattaattaaacctgcaaatactctagttccaaataaagtcacattctcaggttccaggtggacatga

SEQ ID NO: 33 - DNA, 35 bp, Artificial Sequence
  PCR Primer
  primer_bind (1)..(35): PCR Primer
cttggtggacgaagttgctaaagaaattgatgcag

SEQ ID NO: 34 - DNA, 36 bp, Artificial Sequence
  PCR Primer
  primer_bind (1)..(36): PCR Primer
ctgcatcaatttctttagcaacttcgtccacccaag

SEQ ID NO: 35 - DNA, 55 bp, Artificial Sequence
  PCR Primer
  primer_bind (1)..(55): PCR Primer
ctttaagaaggagatataccatgtcacaagaaaggcctacattctaccggcagga

SEQ ID NO: 36 - DNA, 36 bp, Artificial Sequence
  PCR Primer
  primer_bind (1)..(36): PCR Primer
tgatgatgagaaccccccccggactccatttcttct

SEQ ID NO: 37 - DNA, 55 bp, Artificial Sequence
  PCR Primer
  primer_bind (1)..(55): PCR Primer
ctttaagaaggagatataccatgtcacaatcaaaaggtaaaaagcgaaaccctgg

SEQ ID NO: 38 - DNA, 39 bp, Artificial Sequence
  PCR Primer
  primer_bind (1)..(39): PCR Primer
tgatgatgagaaccccccccttagtctccaagaatcagt

SEQ ID NO: 39 - DNA, 1005 bp, Artificial Sequence
  muntant nucleic acid sequence encoding consitutive active MKK6
  misc_feature (619)..(621): Codon substitution GAA substituting for TCT present in wild type sequence
  misc_feature (631)..(633): Codon substitution GAA substituting for ACA present in wild type sequence
atgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggacgaagttgctaaagaaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagacttg

SEQ ID NO: 40 - DNA, 1813 bp, Homo sapiens
gaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaaaaaaaaaaaaa